(19) United States
(12) Patent Application Publication
(10) Pub. No.: US 2010/0189812 A1
(43) Pub. Date: Jul. 29, 2010

(54) AGENT FOR TREATING AND/OR PREVENTING OSTEOPOROSIS, COMPRISING OCEANIC MINERAL COMPONENTS

(75) Inventor: Takeshi OGURA, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: Takeshi Ogura, TOKYO (JP); Midori Nishida, TOKYO (JP)

(21) Appl. No.: 12/358,826

(22) Filed: Jan. 23, 2009

(51) Int. Cl. A61K 33/34

(52) U.S. Cl. 424/630

(57) ABSTRACT

The present invention provides an agent for treating and/or preventing osteoporosis, comprising oceanic mineral components containing essential trace elements. In preparation of the agent, the oceanic mineral complex is allowed to contain as the active ingredient the residue comprising minerals chelated by treating the concentrated seawater containing organic components derived from picoplanktons living in seawater and mineral components chelated by the organic components with charcoal powder and acetic acid and then removing sodium chloride and toxic components.

### TECHNICAL FIELD

The present invention relates to an agent for treating and/or preventing osteoporosis, comprising oceanic mineral components. More specifically, the invention relates to an agent for treating and/or preventing osteoporosis, comprising as active ingredient an oceanic mineral complex obtained by removing sodium chloride and toxic components from concentrated seawater.

### BACKGROUND ART

Recently, relationship between micromineral deficiencies and aging has been attracting attention. The reported similarities between aging symptoms and micromineral deficiencies are shown in Table 1 below.

TABLE 1
Similarities between aging symptoms and
micromineral deficiencies

| | Mineral type |
|---|---|
| | deficiency |
| | in which |
| | causes the same |
| Aging symptoms | symptom |
| Decline in physical strength/ | All minerals |
| Devitalization | |
| Skin atrophy/Balding | Zinc |
| Poor wound healing | Zinc |
| Hypogonadism | Zinc |
| Reduced sense of taste/ | Zinc |
| Loss of appetite | |
| cataractogenesis | Zinc |
| Reduced immune function | Zinc, Copper, |
| | and selenium |
| Onset of depression/dementia | Zinc and Copper |
| ischemic heart disease | Copper, selenium, |
| | zinc and silicon |
| autoimmune disease | Zinc, copper, and |
| | selenium |
| Cancer | Zinc and selenium |
| Abnormal glucose tolerance/ | Chromium and zinc |
| diabetes | |

Pathophysiologic factors in aging include increases in active oxygen and free radicals, immune compromise, circulatory system diseases such as elevation in blood pressure and hyperlipidemia, abnormal glucose tolerance and carcinogenesis. Deficiencies in many kinds of minerals cause same clinical conditions. Especially, free radicals are generated metabolic process in vivo and become a cell-inhibiting factor inhibiting. It has been revealed that increase in such free radicals is involved in mechanisms of aging, life-style-related diseases, and cancers. Generally, an enzyme which can convert such radicals (SOD: superoxide dismutase) works to control toxic actions. In case of micromineral deficiencies in an aged living body, the action of this enzyme is inhibited. In such a case, it is said that natural defense mechanism gets out of order and the living body is devitalized to become more likely to develop lifestyle-related diseases. This is supported by the facts that decrease in SOD enzymes can be redressed by addition of microminerals and that conditions of lifestyle-related diseases such as high blood pressure, hyperlipidemia and diabetes can be improved by addition of microminerals, which is experimentally evidenced. Also, it has been conventionally reported in epidemiologic researches that selenium deficiency or zinc deficiency increases cancer incidence. Thus, micromineral deficiencies are closely related to aging and diseases.

In line with the aging of the society and increasing population of elderly people, the number of patients with osteoporosis is increasing. Osteoporosis afflicts patients by confining them to bed, developing dementia or the like. In osteoporosis, bone resorption(destruction) by osteoclastic cells outpaces bone formation by osteoblast cells. As a result, bone mass decreases and bone becomes fragile. Osteoporosis is roughly categorized into primary osteoporosis and secondary osteoporosis. There is no particular disease causing primary osteoporosis. Primary osteoporosis is caused by dysfunction in bone formation or bone resorption. Secondary osteoporosis is caused by other diseases. It is known that hormone-related diseases such as chronic renal failure, chronic articular rheumatism, hyperthyroidism, hypogonadism and Cushing's syndrome, diabetes, alimentary diseases and the like cause secondary osteoporosis.

Primary osteoporosis is roughly divided into postmenopausal osteoporosis and senile osteoporosis. In case of female patients, a rapidly decline in female hormone estrogen takes place before menopause occurs. With this, a large amount of cytokine which promotes none resorption (destruction) is produced, causing a rapid decline in bone density. Moreover, estrogen has an effect of converting vitamin D into activated vitamin D. Since activated vitamin D has an effect of promoting calcium absorption into the intestine, a decrease in estrogen disturbs absorption of calcium through the intestine and becomes a factor reducing bone mass. Further, the older one is, the more likely osteoporosis is to develop. In case of senile osteoporosis, bone mass decreases due to bone aging and kidney activity declines, which leads to decrease in ability of synthesizing activated vitamin D and decrease in bone mass.

As described above, in osteoporosis, bone mass per volume, i.e. bone density, decreases, and bone becomes light in weight and weak, which leads to easy fractures of bones. That is, bone density increases until the age around 30 when bone formation is more active than bone resorption. After that, when bone formation in vivo becomes insufficient, bone resorption outpaces bone formation and bone density continues to decrease. In case of female bodies, bone density peaks at the age of around 30, and then begins to gradually decrease. It is known that from the age of around 50 when one reaches menopause, decrease in density is accelerated.

It is easier to prevent bone density from decreasing than to recover lost bone density. Therefore, in osteoporosis, generally, prevention is more effective than treatment. Methods for prevention or treatment include appropriate intake of calcium or vitamin D, weight-bearing exercise, and administration of drugs. Among theses, prevention or treatment using drugs, which is useful in maintaining bone density or retarding decrease in bone density, is used in practice appropriately. Examples of drugs used for preventing or treating osteoporosis include calcium agent, estrogen agent, activated vitamin D₃ agent, vitamin K₂ agent, bisphosphonate agent, phosphonate agent, ipriflavone agent, protein anabolic hormone agent, and calcitonin agent.

Calcium agent is expected to have effects in those who lack calcium or in elderly people who do not eat much. Practically, calcium agent is used in combination with activated vitamin D₃ agent, estrogen agent or the like. When calcium agent is used, however, side-effects such as gastrointestinal distress and astriction are often observed. In cases where calcium agent is administered to patients with hyperparathyroidism, a certain type of kidney failure or hypercalcinuria, hypercalcemia may be caused. Estrogen can act directly or indirectly on bones, suppressing bone resorption and maintaining bone density. Estrogen, however, has a problem that it causes menstrual bleeding. For preventing this, care is needed to ingest the agent. Although activated vitamin D₃ agent enhances absorption of calcium through the intestine and suppresses decrease in bone mass, it is known to cause hypercalcemia as side-effect. Vitamin K₂ agent has an effect of preventing bone fractures, and bisphosphonate agent suppresses decrease in bone density. These agents, however, sometimes cause gastrointestinal distresses as side-effects. Ipriflavone agent, which is said to have both effects of accelerating bone formation and suppressing bone resorption, often causes gastrointestinal distress. Protein anabolic hormone agent is recognized as having an effect of increasing bone density. Since it is hormone-based agent, it has many side effects and care must be taken in using the agent and patient types which the agent may be used for are limited. Calcitonin agent has an effect of suppressing bone resorption by osteoclastic cells, to thereby suppress decrease in bone density. Since the agent is administered intramuscularly, the burden on the patient becomes too much if it is used for a long term. Moreover, it is known that effects of calcitonin are lessened if it is used for a long time.

### DISCLOSURE OF INVENTION

### Problems to be Solved by Invention

As described above, there are often unignorable side-effects or risks or heavy burdens on patients in cases using conventional drugs for preventing or treating osteoporosis. Moreover, even if one agent is necessary for treating symptoms or clinical conditions of one patient, the agent cannot be used for the patient in some cases. The objective of the present invention is to provide an agent for treating and/or preventing osteoporosis effectively by using oceanic mineral components which components contain essential trace elements, which agent can solve or alleviate one or more of the above problems in conventional agents.

### Means for Solving the Problem

Generally, when one enters his middle-life, the balance between microminerals in the body collapses due to decreases in amounts of intake and absorption or disorders in metabolism or excretion process, which leads to chronic deficiency in microminerals. Accordingly, intakes of microminerals play an important role in maintaining health and preventing diseases for people entering middle-age generation.

Minerals springing from thermal deposits existing in the sea bed and minerals flowing in from land for 3.6 billion years since the earth was born are dissolved in seawater. All the trace elements related to aging symptoms as above described are contained in seawater. Components constituting the blood or body fluid flowing in the human body are said to be closely similar with components of primordial sea water. It has been already assumed that microminerals are essential elements for cellular-level metabolism which supports life activity of the living body.

Recently, developments have been made on methods required for analyzing elements which exist at only a low concentration in human body. Further, functions of various trace elements have been brought out by biochemical studies. Thus, essentiality of trace elements in human body has been confirmed.

Main elements constituting the living body as proteins, nucleic acids or blood are hydrogen (H), oxygen (O), carbon (C), and nitrogen (N), and a small amount of phosphorus(P) and sulfur(S) is also contained. Moreover, elements constituting bone and body fluid are calcium (Ca), Sodium (Na), Potassium (K), magnesium (Mg), and chloride (Cl). Together with these major elements, there are essential trace elements in the living body, such as iron (Fe), zinc (Zn), copper (Cu), arsenic (As), chromium (Cr), cobalt (Co), selenium (Se), manganese (Mn), molybdenum, silicon (Si), fluorine (F), vanadium (V), nickel (Ni), tin (Sn), and iodine (I).

All of these essential trace elements are transition elements whose charge states easily change. Many of them act as enzymes which catalyze oxidation reduction reaction where electrons are given or received or as components for coenzymes in the living body. Recently, functions of theses trace elements have been clarified by finding that deficiencies in essential trace elements such as chromium and zinc cause disorders in sugar metabolism which requires insulin or in metabolism of protein and nucleic acid (Saishin-Igaku, 45, 808,(1990)). Also, as for diabetes, effects of sea water minerals on diabetic mice have been confirmed by experiments (Japan Medical Journal, Vol. 3675, 1-Oct.-1994).

The present inventors have made extensive studies on how oceanic minerals contained in sea water act in the living body and have already proposed an agent for treating allergies comprising as an active ingredient a mineral complex (Marina Calcium Mineral; hereinafter abbreviated as MCM: a product of Kaiyo Kagaku Kenkyukai) containing major elements and trace elements, which complex is obtained by removing sodium chloride from concentrated seawater as much as possible and then removing toxic components such as mercury, which agent is effective for treating hepatitis, high blood pressure, tumor, atopic dermatitis, nasal inflammation and the like (Japanese Patent No. 3247620).

As for osteoporosis, conventional drugs currently used for treating and/or preventing osteoporosis have problems of side-effects, risks, administration methods, burdens on patients and poor potentiality of applying to various types of patients, as above described. Moreover, the most important effect of increasing bone mass by these drugs is not so great as desired and is in a level that the bone mass does not decrease. The present inventors have made extensive studies with a view to using the above-mentioned mineral complex (MCM) containing major elements and trace elements in an agent for treating and/or preventing osteoporosis free from side-effects and burdens on patients and essentially applicable to any patient, and have found out that MCM can be expected to increase bone mass and can be an effective agent for treating and/or preventing osteoporosis.

That is, the present invention provides an agent for treating and/or preventing osteoporosis, as follows.
1. An agent for treating and/or preventing osteoporosis comprising as an active ingredient an oceanic mineral complex (MCM) consisting of residue which is obtained by treating concentrated seawater containing organic components derived from picoplanktons living in seawater and mineral components chelated by the organic components with acetic acid and then removing sodium chloride and toxic components therefrom.
2. The agent for treating and/or preventing osteoporosis comprising an oceanic mineral complex according to 1, wherein calcium which is required for bone formation is contained in an amount enhanced by allowing the mineral-containing concentrated seawater to react with calcium acetate.
3. The agent for treating and/or preventing osteoporosis comprising an oceanic mineral complex according to 1 or 2, wherein calcium which is required for bone formation is contained in an amount enhanced by calcining calcium carbonate, adding acetic acid to the calcined calcium carbonate to thereby convert it into calcium acetate and then adding the calcium acetate to the heated concentrated seawater.
4. The agent for treating and/or preventing osteoporosis according to any one of 1 to 3, wherein the oceanic mineral complex (MCM) is obtained by concentrating raw material seawater by heat, then removing sodium chloride from the concentrated seawater and filtrating off toxic components such as mercury as solidified matters.
5. The agent for treating and/or preventing osteoporosis according to any one of 1 to 4, wherein the oceanic mineral complex (MCM) is obtained by concentrating raw material seawater by heat, then adding acetic acid and charcoal powder to the seawater, heating followed by cooling, filtering off mainly sodium chloride and toxic components such as mercury as solidified matters to thereby obtain filtrate containing MCM.
6. The agent for treating and/or preventing osteoporosis according to 4 or 5, wherein the oceanic mineral complex (MCM) consist of residue which is obtained by repeating the following operations (1) to (3):
(1) adding acetic acid and charcoal powder to the sea water;
(2) heating followed by cooling; and
(3) filtrating off solidified matters, and further concentrating the final filtrate.
7. The agent for treating and/or preventing osteoporosis according to any one of 1 to 6, wherein the oceanic mineral complex (MCM) comprises crystalline solid powder containing minerals chelated by adding charcoal powder and acetic acid to the concentrated seawater containing organic components derived from picoplanktons living in seawater and mineral components chelated by the organic components, and the mixture is heated and then to thereby remove precipitated sodium chloride and toxic components.
8. The agent for treating and/or preventing osteoporosis according to 7, wherein the oceanic mineral complex (MCM) comprising crystalline solid powder containing the minerals contains 20 to 30% by mass of organic components derived from planktons.
9. The agent for treating and/or preventing osteoporosis according to any one of 1 to 8, wherein the oceanic mineral complex (MCM) contains trace elements essentially required in the living body in addition to major elements of calcium (Ca), sodium (Na), potassium (K) and magnesium (Mg).
10. The agent for treating and/or preventing osteoporosis according to 9, wherein the oceanic mineral complex (MCM) contains as the trace elements essentially required in the living body at least one of iron (Fe), zinc (Zn), copper (Cu), chromium (Cr), selenium (Se), manganese (Mg) and silicon (Si).
11. The agent for treating and/or preventing osteoporosis according to any one of 1 to 10, which is orally administered.
12. The agent for treating and/or preventing osteoporosis according to 11, wherein the oceanic mineral complex (MCM) is used as a solid composition, a liquid composition or other states of composition used for oral administration.

### Effects of Invention

The oceanic mineral complex (MCM) according to the present invention contains trace elements essentially required in the living body in addition to major elements of calcium (Ca), sodium (Na), potassium (K) and magnesium (Mg), which consist of components obtained by removing toxic components such as sodium chloride and organic mercury from concentrated seawater. The present invention is excellent in that the agent for treating and/or preventing osteoporosis can be administered basically to all types of patients to sufficiently increase bone mass without problems of side-effects and burdens on patients and thus be effectively used for treating, preventing and improving the symptoms of the disease.

### BEST MODE FOR CARRYING OUT INVENTION

Hereinafter, the present invention is described in detail.

### [Production Method of the Mineral Complex (MCM) Containing Major Elements and Trace Elements Essentially Required in the Living Body]

The agent for treating and/or preventing osteoporosis according to the present invention comprises as the active ingredient the oceanic mineral complex (MCM) consisting of residue containing chelated minerals obtained by treating concentrated seawater containing organic components derived from picoplanktons living in seawater and mineral components chelated by the organic components with acetic acid and removing sodium chloride and toxic components therefrom. In the present invention, the oceanic mineral complex can be prepared with calcium which is required for bone formation contained in an amount enhanced by adding calcium acetate to the concentrated seawater containing the minerals to react with each other. In a preferred embodiment, calcium carbonate is calcined and then acetic acid is added thereto to thereby convert it into calcium acetate. By adding this to heated concentrated seawater, the oceanic mineral complex can be prepared with an enhanced amount of calcium required for bone formation. To concentrated seawater containing the minerals, generally 10-30%, preferably 20% of calcium carbonate is added in volume ratio and the mixture is calcined generally at about 300-500° C., preferably about 400° C. To this, generally 90-100%, preferably 98-99.9% grade acetic acid is added, to convert it into calcium acetate. The resultant is added to the concentrated seawater heated generally at a heat source temperature of about 100-200° C., preferably at about 150° C. After reaction starts, the mixture is left standing generally for about 24 to 120 hours, preferably about 48 to 96 hours, more preferably about 72 hours, to thereby complete the reaction calmly. It is preferable that the amount of calcium required for bone formation contained in the oceanic mineral complex be enhanced. MCM, which is the active ingredient in the present invention, is prepared, for example, by using as raw material seawater pumped up from a clean seawater region of about 80 to 120 meters deep under the sea. Table 2 shows main elements contained in 18 liters of sea water which was pumped up from depth of about 100 meters under the sea surface in a typical black current region (off Oarai coast, Japan) and proportion.

TABLE 2
Table 2: Amount of each element contained in 18 L of
seawater sampled (μg)

| Element | Weight (μg) |
|---|---|
| Sodium (Na) | 49,500,000 |
| Magnesium (Mg) | 6,125,000 |
| Sulfur (S) | 4,240,000 |
| Potassium (K) | 1,880,000 |
| Calcium (Ca) | 1,790,000 |
| Carbon (C) | 132,000 |
| Silicon (Si) | 14,130 |
| Nitrogen (N) | 2,350 |
| Phosphorus (P) | 330 |
| Zinc (Zn) | 150 |
| Iron (Fe) | 150 |
| Chromium (Cr) | 470 |
| Selenium (Se) | 14 |

This raw material seawater is heated under normal pressure or reduced pressure, to thereby concentrate it until the volume becomes reduced to about 1/60 of the initial volume. Next, sodium chloride is removed from this concentrated water as much as possible. By adding 99% grade acetic acid of the almost same volume as the volume of the concentrated water and about 0.5% by weight of charcoal powder to the concentrated seawater, heating the mixture to about 400° C. and then cooling it to −12° C., components containing sodium chloride mainly, mercury and the like are solidified. This solid product is filtered. To the obtained filtrate, the operations of (1) adding acetic acid and charcoal powder, (2) heating followed by cooling and (3) filtrating off solid matters, in the same manner as described above, are repeated about four times, to obtain a final filtrate. In the thus obtained oceanic mineral complex (MCM) consisting of residue solid product, components are chelated by picoplanktons, which are organic components of seawater, and MCM as is can be used in the agent for treating and/or preventing osteoporosis.

Table 3 shows elements contained in 1 g of the crystalline solid product (MCM) chelated by organic substances present in seawater with sodium chloride and toxic components having been removed therefrom. It is preferred that in the agent for treating and/or preventing osteoporosis, the crystalline solid powder (MCM) containing minerals about 20 to 30% by mass of organic components derived from planktons, particularly preferably about 25% by mass.

TABLE 3
Table 3: Major elements and trace elements contained in 1 g of MCM

| Components | weight (μg) |
|---|---|
| Water (H₂O) | 242,700 |
| Sodium (Na) | 54,300 |
| Potassium (K) | 81,600 |
| Calcium (Ca) | 310,500 |
| Magnesium (Mg) | 1,700 |
| Organic substances | 253,400 |
| (C, H, O, S, P, N) | |
| Chloride(Cl) | 35,300 |
| Silicon (Si) | 125 |
| Iron (Fe) | 125 |
| Copper (Cu) | 8 |
| Chromium (Cr) | 250 |
| Manganese (Mn) | 10 |
| Zinc (Zn) | 60 |
| Selenium (Se) | 16 |
| Cadmium (Cd) | Not detected |
| Lead (Pb) | Not detected |
| Organic mercury (Hg) | Not detected |

### [Toxicity]

It has been confirmed that toxicity of the oceanic mineral complex (MCM) according to the present invention is so sufficiently low that it can be safely used for medical purposes.

### [Application to Medical Products]

The agent for treating and/or preventing osteoporosis according to the present invention comprises an oceanic mineral complex, MCM, which is residue containing chelated minerals, obtained by subjecting concentrated seawater containing organic components derived from picoplanktons present in seawater and minerals chelated by the organic components to treatment with acetic acid and removing sodium chloride and toxic components therefrom. The oceanic mineral complex (MCM) according to the present invention is, as already known, effective when used in treating diabetes, hepatitis, high pressure, tumor, atopic dermatitis, nasal inflammation and other allergic diseases, and also in treating and preventing osteoporosis. The oceanic mineral complex (MCM) according to the present invention is used generally by oral administration, for the purpose of treating/preventing osteoporosis. The dosage amount depends on age, weight, symptoms, target in treatment/prevention, administration method, administration time and so forth. Generally, a single dose for adult is within a range of 300 to 400 mg and the number of doses per day is 1 to several times.

When the oceanic mineral complex (MCM) according to the present invention is administered, it is prepared into a solid composition, liquid composition or other forms for oral administration. Examples of solid forms for oral administration include tablet, pill, capsule, powder, and granule. Examples of liquid forms for oral administration include opalizer, solution, syrup, and elixir and may further contain inactive diluent (for example, purified water and ethanol). In this composition, adjuvants such as moisturizer and suspension agent, and sweetening agent, flavor, fragrance, and/or antiseptic may be contained.

### EXAMPLE

Hereinafter, effects of the agent for treating and preventing osteoporosis are explained based on Example. The present invention is, however, by no means limited to Example.

### Preparation of MCM:

18 L of clean seawater was sampled out from a black current region (off Oarai coast, Japan) about 100 meters deep under the sea surface. (Components are shown in Table 2). This was concentrated by heating and then treated with charcoal powder and acetic acid. Sodium chloride and components such as organic mercury which are harmful to the living body were removed. The operation of heating and removing by freezing was repeated, so that the oceanic minerals were condensed and crystallized, to thereby obtain a powdery solid (MCM) as shown in Table 3.

The above prepared MCM was orally administered to a 80-year-old (male) patient during his treatment period. His bone density values before treatment and after treatment were compared. The results are shown in Table 4. Table 4 also shows treatment results of a 74-year-old (female) patient and healthy people of a 70-year-old (male) and 69-year-old (female). Here, the values shown by percent (%) are percentage when compared with the average bone density of each age. The measurement on bone density was made by using an X-ray bone density measurer DTX-200 (manufactured by TOYO MEDIC CO., LTD.) for the 80-year-old patient and the healthy 70-year-old (male) and 69-year-old (female) and by bone mineral quantitation using X-ray for the 74-year-old (female) patient.

TABLE 4

| Patient's | | |
|---|---|---|
| age and | | |
| sex | Before treatment | After treatment |
| 80 | 70% | 75% |
| (male) | Measurement date: | Measurement date: |
| | Aug. 5, 2004 | Jan. 20, 2006 |
| 74 | 76% | 86% |
| (female) | Measurement date: | Measurement date: |
| | Nov. 19, 2004 | Jan. 26, 2006 |

| | |
|---|---|
| 70 | 100% |
| (male) | Measurement date: Feb. 22, 2006 |
| 69 | 103% |
| (female) | Measurement date: Feb. 22, 2006 |

As seen in Table 4, the bone density of the 80-year-old patient (male) increased by 5%, from 70% (compared with the average bone density of the age) before treatment to 75% after treatment. Also, with respect to the 74-year-old (female), her bone density increased by 10%, from 76% (compared with the average bone density of the age) before treatment to 86% after treatment.

What is claimed is:

1. An agent for treating and/or preventing osteoporosis comprising as an active ingredient an oceanic mineral complex (MCM) consisting of residue which is obtained by treating concentrated seawater containing organic components derived from picoplanktons living in seawater and mineral components chelated by the organic components with acetic acid and then removing sodium chloride and toxic components therefrom.

2. The agent for treating and/or preventing osteoporosis comprising an oceanic mineral complex according to claim 1, wherein calcium which is required for bone formation is contained in an amount enhanced by allowing the mineral-containing concentrated seawater to react with calcium acetate.

3. The agent for treating and/or preventing osteoporosis comprising an oceanic mineral complex according to claim 1, wherein calcium which is required for bone formation is contained in an amount enhanced by calcining calcium carbonate, adding acetic acid to the calcined calcium carbonate to thereby convert it into calcium acetate and then adding the calcium acetate to the heated concentrated seawater.

4. The agent for treating and/or preventing osteoporosis according to claim 1, wherein the oceanic mineral complex (MCM) is obtained by concentrating raw material seawater by heat, then removing sodium chloride from the concentrated seawater and filtrating off toxic components such as mercury as solidified matters.

5. The agent for treating and/or preventing osteoporosis according to claim 1, wherein the oceanic mineral complex (MCM) is obtained by concentrating raw material seawater by heat, then adding acetic acid and charcoal powder to the seawater, heating followed by cooling, filtering off mainly sodium chloride and toxic components such as mercury as solidified matters to thereby obtain filtrate containing MCM.

6. The agent for treating and/or preventing osteoporosis according to claim 4, wherein the oceanic mineral complex (MCM) consist of residue which is obtained by repeating the following operations (1) to (3):
(1) adding acetic acid and charcoal powder to the sea water;
(2) heating followed by cooling; and
(3) filtrating off solidified matters, and further concentrating the final filtrate.

7. The agent for treating and/or preventing osteoporosis according to claim 5, wherein the oceanic mineral complex (MCM) consist of residue which is obtained by repeating the following operations (1) to (3):
(1) adding acetic acid and charcoal powder to the sea water;
(2) heating followed by cooling; and
(3) filtrating off solidified matters, and further concentrating the final filtrate.

8. The agent for treating and/or preventing osteoporosis according to claim 1, wherein the oceanic mineral complex (MCM) comprises crystalline solid powder containing minerals chelated by adding charcoal powder and acetic acid to the concentrated seawater containing organic components derived from picoplanktons living in seawater and mineral components chelated by the organic components, and the mixture is heated and then to thereby remove precipitated sodium chloride and toxic components.

9. The agent for treating and/or preventing osteoporosis according to claim 8, wherein the oceanic mineral complex (MCM) comprising crystalline solid powder containing the minerals contains 20 to 30% by mass of organic components derived from planktons.

10. The agent for treating and/or preventing osteoporosis according to claim 1, wherein the oceanic mineral complex (MCM) contains trace elements essentially required in the living body in addition to major elements of calcium (Ca), sodium (Na), potassium (K) and magnesium (Mg).

11. The agent for treating and/or preventing osteoporosis according to claim 10, wherein the oceanic mineral complex (MCM) contains as the trace elements essentially required in the living body at least one of iron (Fe), zinc (Zn), copper (Cu), chromium (Cr), selenium (Se), manganese (Mg) and silicon (Si).

12. The agent for treating and/or preventing osteoporosis according to claim 1, which is orally administered.

13. The agent for treating and/or preventing osteoporosis according to claim 12, wherein the oceanic mineral complex (MCM) is used as a solid composition, a liquid composition or other states of composition used for oral administration.
